(19) United States
(12) Patent Application Publication
(10) Pub. No.: US 2007/0043418 A1
(43) Pub. Date: Feb. 22, 2007

(54) Hybrid lumen-supporting stents having self-expanding end segments

(75) Inventors: Michael J. Lee, Santa Rosa, CA (US); Richard L. Klein, Santa Rosa, CA (US)

Correspondence Address: PRESTON GATES & ELLIS LLP;ATTN: C. RACHAL WINGER, 925 FOURTH AVE, SUITE 2900, SEATTLE, WA, 98104-1158, US

(73) Assignee: Medlogics Device Corporation, Santa Rosa, CA (US)

(21) Appl. No.: 11/207,483

(22) Filed: Aug. 19, 2005

(51) Int. Cl. A61F 2/06

(52) U.S. Cl. 623/1.2

(57) ABSTRACT

A stent and stent deployment system and related methods are disclosed wherein the stent has at least one end segment comprised of a self-expanding material attached to a body portion comprised of a biocompatible material. The stent deployment system comprises a balloon catheter having a stent disposed over the balloon. In one embodiment a stent's end segments extend beyond the proximal and distal ends of the balloon such that they are able to stent a portion of a vessel not expanded or damaged by balloon expansion. This stenting beyond area expanded or damaged by balloon expansion provides for a less abrupt transition between stented and unstented portions of a vessel.

### FIELD OF THE INVENTION

This invention relates to improved implantable stents for the treatment of stenoses in coronary or peripheral vessels in humans. More specifically, the invention relates to an improved implantable stent with self-expanding end segments that provide for a less abrupt transition between stented and unstented portions of a vessel and also improve stent flexibility.

### BACKGROUND OF THE INVENTION

Cardiovascular disease, including atherosclerosis, is the leading cause of death in the United States. The medical community has developed a number of methods and devices for treating coronary heart disease, some of which are specifically designed to treat the complications resulting from atherosclerosis and other forms of coronary artery narrowing.

An important development for treating atherosclerosis and other forms of coronary narrowing is percutaneous translumenal coronary angioplasty, hereinafter referred to as “angioplasty.” The objective of angioplasty is to enlarge the lumen (inner tubular space) of the affected coronary artery by radial hydraulic expansion. The procedure is accomplished by inflating a balloon within the narrowed lumen of the affected artery. Radial expansion of the affected artery occurs in several different dimensions, and is related to the nature of the plaque narrowing the vessel lumen. Soft, fatty plaque deposits are flattened by the balloon, while hardened deposits are cracked and split to enlarge the lumen. The wall of the artery itself is also stretched when the balloon is inflated.

Unfortunately, while the affected artery can be enlarged thus improving blood flow, in some instances the vessel re-occludes chronically (“restenosis”), or closes down acutely (“abrupt reclosure”), negating the positive effect of the angioplasty procedure. Restenosis or abrupt reclosure frequently necessitates repeat angioplasty or open heart surgery. While such restenosis or abrupt reclosure does not occur in the majority of cases, it occurs frequently enough that such complications comprise a significant percentage of the overall failures of the angioplasty procedure, for example, twenty-five to thirty-five percent of such failures.

To lessen the risk of restenosis or abrupt reclosure, various devices have been proposed for mechanically keeping the affected vessel open after completion of the angioplasty procedure. Such endoprostheses (generally referred to as “stents”), are typically inserted into the vessel, positioned across the lesion or stenosis, and then expanded to keep the passageway clear. The stent provides a scaffold which overcomes the natural tendency of the vessel walls of some patients to restenose or undergo abrupt reclosure, thus maintaining the openness of the vessel and resulting blood flow.

In order to prevent restenosis or abrupt reclosure within a vessel, a stent must have adequate radial strength to hold the vessel open. To achieve this required radial strength, many stents are constructed of a stiff and inflexible material such as stainless steel alloys. Further, these stents often are constructed so that, upon deployment, they are expanded beyond their elastic limit. Expanding a material past its elastic limit causes it to enter its plastic phase where it becomes stiffer and less flexible.

The procedure of angioplasty and characteristics of stents just described often result in an abrupt transition from stented to unstented portions of a vessel that can exacerbate the physiological trauma found at an implant site. Specifically, when a balloon-expandable stent is implanted in a vessel, the balloon expansion expands the vessel beyond its normal circumference. This expansion alone damages the vessel and causes a transition site from the stented to unstented portion of the vessel. Further, the area of the vessel where the stent is placed remains larger than the surrounding area of the vessel even after the balloon has been removed. The increased size of the vessel, its resulting damage, along with the sudden end of a stiff and inflexible implanted stent, all contribute to creating an abrupt transition from the stented to the unstented area of the vessel. Such an abrupt transition can be problematic for various reasons. It may impede blood flow at the transition point, increase inflammation at the stent-vessel interface, provide a place for platelets to adhere and for plaque to build up and lead to immune activation in the area. Thus, while stents and stent applications have been found to work well in a number of patients, there is still room for improvement. Specifically, a need exists for a stent that provides for a less abrupt transition between stented and unstented portions of a vessel. Accordingly, the stents, systems and methods of the present invention provide embodiments that reduce transition abruptness between stented and unstented portions of a vessel.

### SUMMARY OF THE INVENTION

The stents of the present invention provide for less abrupt transitions from stented to unstented portions of vessels as compared with the use of conventional stents. Providing for a less abrupt transition from stented to unstented portions of a vessel can reduce the risk of restensosis or abrupt reclosure after an angioplasty procedure. The present invention provides for a less abrupt transition from stented to unstented portions of a vessel by providing a balloon-expandable stent. Connected to the ends of the balloon-expandable stent are self-expanding end segments that can, in one embodiment, extend beyond the ends of the inflatable balloon. During deployment of the stent, the self-expanding segments can be kept compressed under sleeves. When the stent is positioned and the balloon expanded, expansion of the balloon deploys the body of the stent and also pushes the sleeves back, thus allowing the self-expanding segments to deploy beyond the area of the vessel expanded by the balloon. These self-expanding segments thus stent an area of the vessel beyond the damaged portion leading to an improved transition between the damaged and undamaged (i.e. stented and unstented) portions of the vessel.

One embodiment of the present invention includes a stent comprising a body portion wherein the body portion has a first end and a second end and wherein the first end is attached to a first end segment and wherein the body comprises a first material and the first end segment comprises a second material and wherein the second material is a self-expanding material.

In another embodiment of the stents of the present invention, the first material is selected from the group consisting of stainless steel, titanium, gold, cobalt alloys, magnesium, platinum, platinum alloys and tantalum alloys. In another embodiment of the stents of the present invention, the second material is a self-expanding material which is a metal alloy selected from the group consisting of CuZnAl, CuAlNi, spring temper stainless steel and nickel-titanium alloys.

In another embodiment of the stents of the present invention, the second end of the body portion is attached to a second end segment wherein the body portion comprises a first material and the second end segment comprises a second material and wherein the second material is a self-expanding material.

In another embodiment of the stents of the present invention, the stent further comprises a coating comprising at least one biocompatible polymer or metal and, within the biocompatible polymer or metal, a bioactive agent selected from the group consisting of antineoplastic agents, antinflammatory agents, antiplatelet agents, anticoagulant agents, antifibrin agents, antithromobin agents, antimitotic agents, antibiotic agents, antiproliferative agents, antioxidant substances, calcium channel blockers, colchicine fibroblast growth factor antagonists, histamine antagonists, 3-hydroxy-3-methylglutaryl coenzyme A (HMG-CoA) reductase inhibitors, monoclonal antibodies, phosphodiesterase inhibitors, prostaglandin inhibitors, platelet-derived growth factor antagonists, serotonin inhibitors, steroids, and thioprotease inhibitors.

The present invention also includes systems for deploying a stent comprising one or more end segments. In one embodiment of the systems of the present invention, the system comprises a catheter having a catheter shaft, the catheter shaft having a distal end and a proximal end; an inflatable balloon portion disposed within the distal end of the catheter shaft and in fluid communication with the catheter shaft such that the inflatable balloon portion can be inflated; the inflatable balloon portion having a proximal end and a distal end; and a stent disposed over the inflatable balloon portion, the stent comprising a body portion wherein the body portion has a first end and a second end and wherein the body portion has a first end and a second end and wherein the first end is attached to a first end segment and wherein the body comprises a first material and the first end segment comprises a second material and wherein the second material is a self-expanding material. In another embodiment of the systems of the present invention, the second end of the body portion is attached to a second end segment wherein the body portion comprises a first material and the second end segment comprises a second material and wherein the second material is a self-expanding material.

In another embodiment of the stents of the present invention, the first material is selected from the group consisting of stainless steel, titanium, gold, cobalt alloys, magnesium, platinum, platinum alloys and tantalum alloys. In another embodiment of the stents of the present invention, the second material is a self-expanding material which is a metal alloy selected from the group consisting of CuZnAl, CuAlNi, spring temper stainless steel and nickel-titanium alloys.

In another embodiment of the systems of the present invention, the body portion of the stent further comprises a coating comprising at least one biocompatible polymer or metal and within the biocompatible polymer or metal, a bioactive agent selected from the group consisting of antineoplastic agents, antinflammatory agents, antiplatelet agents, anticoagulant agents, antifibrin agents, antithromobin agents, antimitotic agents, antibiotic agents, antiproliferative agents, antioxidant substances, calcium channel blockers, colchicine fibroblast growth factor antagonists, histamine antagonists, HMG-CoA reductase inhibitors, monoclonal antibodies, phosphodiesterase inhibitors, prostaglandin inhibitors, platelet-derived growth factor antagonists, serotonin inhibitors, steroids, and thioprotease inhibitors.

In another embodiment of the systems of the present invention, the first end segment of the stent can extend beyond the proximal end of the balloon and the second end segment can extend beyond the distal end of the balloon. In another embodiment of the systems of the present invention, the first end segment and the second end segment are held in contact with the catheter by retaining sleeves. In another embodiment of the systems of the present invention, the retaining sleeves are affixed to the catheter using an adhesive.

The present invention also includes methods of providing a system for deploying a stent with one or more self-expanding end segments. In one embodiment of the methods of the present invention, the method provides a system for deploying a stent comprising providing a catheter with a catheter shaft including a distal end and a proximal end; providing an inflatable balloon portion disposed within the distal end of the catheter shaft and in fluid communication with the catheter shaft such that the inflatable balloon portion can be inflated, the inflatable balloon portion having a proximal end and a distal end; disposing a stent over the inflatable balloon portion, the stent comprising a body portion disposed between a first end segment and a second end segment wherein the body portion has a first end and a second end and wherein the body portion has a first end and a second end and wherein the first end is attached to a first end segment and wherein the body comprises a first material and the first end segment comprises a second material and wherein the second material is a self-expanding material. In another embodiment of the systems of the present invention, the second end of the body portion is attached to a second end segment wherein the body portion comprises a first material and the second end segment comprises a second material and wherein the second material is a self-expanding material.

In another embodiment of the methods of the present invention, the body portion comprises a metal coating containing the bioactive material paclitaxel.

In another embodiment of the methods of the present invention, the first end segment of the stent extends beyond the proximal end of the balloon and the second end segment extends beyond the distal end of the balloon. In another embodiment of the methods of the present invention, the first end segment and the second end segment are held in contact with the catheter by silicone retaining sleeves. In another embodiment of the methods of the present invention, a lubricant is associated with the retaining sleeves. In another embodiment of the methods of the present invention, the retaining sleeves are affixed to the catheter using an urethane-based adhesive.

In another embodiment of the stents of the present invention, the first material is selected from the group consisting of stainless steel, titanium, gold, cobalt alloys, magnesium, platinum, platinum alloys and tantalum alloys. In another embodiment of the stents of the present invention, the second material is a self-expanding material which is a metal alloy selected from the group consisting of CuZnAl, CuAlNi, spring temper stainless steel and nickel-titanium alloys.

### BRIEF DESCRIPTION OF THE DRAWINGS

FIGS. 1A-1D depict various exemplary stent configurations that can be used as stent body portions in accordance with the present invention.

FIGS. 2A and 2B depict the stent body portion of FIG. 1A with self-expanding end segments attached to it in accordance with the present invention.

FIG. 3 depicts a stent of the present invention (including a stent body portion with self-expanding end segments) over a balloon catheter with the self-expanding nitinol end segments retained under sleeves.

FIG. 4 depicts expansion of the balloon deploying the body of the stent and pushing the sleeves back so that the self-expanding end segments deploy freely.

### DETAILED DESCRIPTION OF THE INVENTION

Any balloon-expandable stent can be used as the main body of the stent in accordance with the present invention. For non-limiting examples, see United States Patent Numbers (USPN) U.S. Pat. No. 5,292,331 to Boneau, U.S. Pat. No. 5,135,536 to Hilstead, U.S. Pat. No. 5,158,548 to Lau et al., and the references cited therein. The present invention is applicable to all known stent configurations, and it will be readily apparent from the following discussion of several exemplary configurations how the invention can be applied to any other type of stent construction.

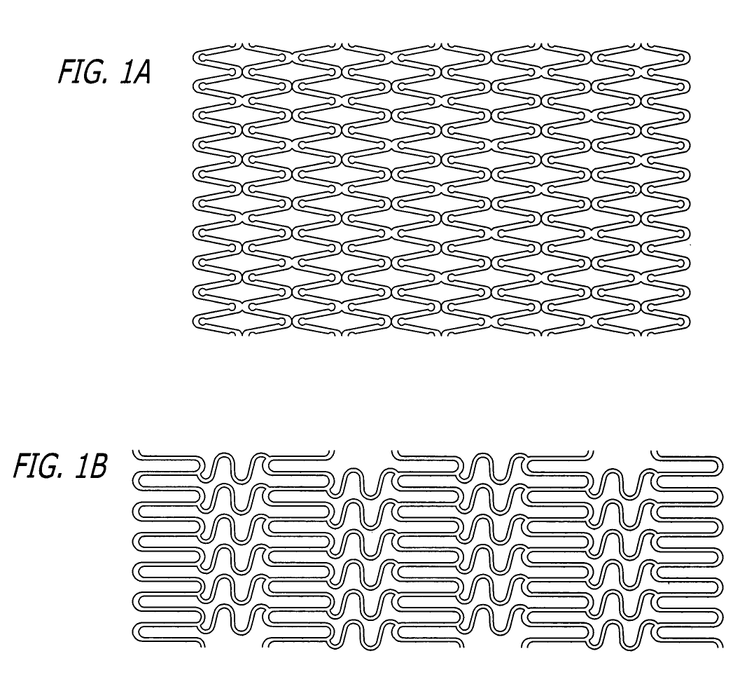

FIGS. 1A-1D depict four different exemplary stent configurations useful in accordance with the teaching of the present invention. The stents of the present invention can have more or less undulations than are shown in FIGS. 1A-1D but these simplified depictions are sufficient to illustrate the present invention. In these examples depicted in FIGS. 1A-1D, the stent bodies are unitary, meaning that they are fashioned from a single piece of material. For example, the stent bodies are cut to an appropriate length from appropriate material tubing. The tubing is then laser cut to form the strut pattern. In another embodiment of the present invention, the stent bodies need not be unitary but instead can consist of individual segments joined by welding, soldering, adhesive bonding, mechanical fastening, or in any other suitable way.

Once a stent body configuration is chosen, self-expanding material such as, but not limited to, nitinol end segments, are attached to one or more ends of the stent body portion. Again, these self-expanding material end segments can be unitary or formed of individual segments and can be attached to the stent body by welding, soldering, adhesive bonding, mechanical fastening, or in any other suitable means known to those having ordinary skill in the art of catheter and vascular stent system design. The self-expanding materials used in accordance with the teachings of the present invention include materials made from, without limitation, CuZnAl, CuAlNi, spring temper stainless steel, and nickel-titanium alloys such as nitinol; however, nitinol will be used as the exemplary embodiment and thus will be referred to exclusively hereinafter when referring to self-expanding materials. Note, however, that the use of the term “nitinol” is merely for convenience and should not be considered limiting. Persons having ordinary skill in the art will realize that materials demonstrating self-expandability are materials that have almost rubber-like flexibility and recover from large amounts of deformation upon heating or cooling.

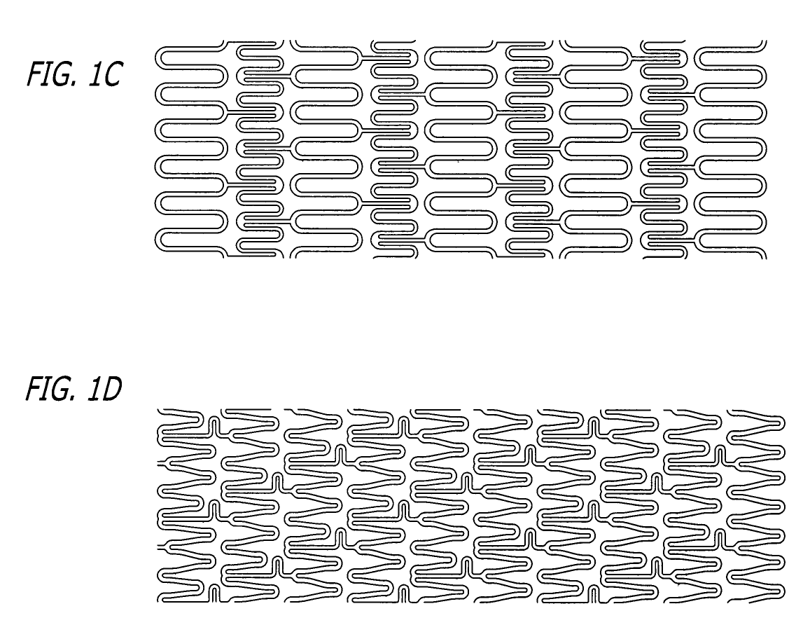

FIGS. 2A and 2B depict the stent body of FIG. 1A with self-expanding end segments attached to it. FIG. 2A depicts the stent body portion of FIG. 1A with a self-expanding end segments 60 and 70 attached to each end of the stent body 80. As can be seen in FIG. 2A the end segments can, but need not, have the same number of sections between them. FIG. 2B depicts the stent body of FIG. 1A with a self-expanding end segment 90 attached to one end of the stent body 80. In one example, this embodiment of the stents of the present invention can be useful when more than one stent is placed at a particular treatment site. For instance, a stent with one end segment can be loaded onto a balloon catheter such that the self-expanding end segment is on the balloon catheter's distal end and can be deployed at a treatment site. Next, a stent with no self-expanding end segments can be deployed at the treatment site in a delivery position that is proximal to the previously-deployed stent. Finally, a stent with one end self-expanding segment can be loaded onto a balloon catheter such that the self-expanding end segment is on the balloon catheter's proximal end and can be deployed at the treatment site in a delivery position proximal to the previously-deployed stents. In this manner, the treatment site as a whole would have a self-expanding end segment at each end while no individual stent includes two self-expanding end segments.

Self-expanding nitinol end segments are included on the ends of the stent bodies of the present invention to provide for a less abrupt transition between stented and unstented portions of vessels than that that would be observed with the use of a conventional stent without these self-expanding nitinol end segments. Specifically, conventional prior art stents are deployed by inflating a balloon attached to the distal end of a catheter on which a stent is compressed. During deployment, balloon inflation expands the stent against a vessel's lumenal wall. This inflation can result in damaging the stented area. Further, because the stent ends at approximately the same location as the balloon catheter, an abrupt transition is created between the stented and unstented portions of the vessel. In the present invention, self-expanding nitinol end segments are included on the ends of the stent body. These self-expanding nitinol end segments expand without balloon expansion and, in one embodiment, can stent a portion of the vessel beyond that expanded and damaged by the balloon expansion used to deploy the stent body. These self-expanding nitinol end segments allow a certain degree of vascular recoil, above that of the stent body, and thus create a less abrupt transition between stented and unstented portions of a vessel and thus can reduce the risk of restenosis or abrupt reclosure.

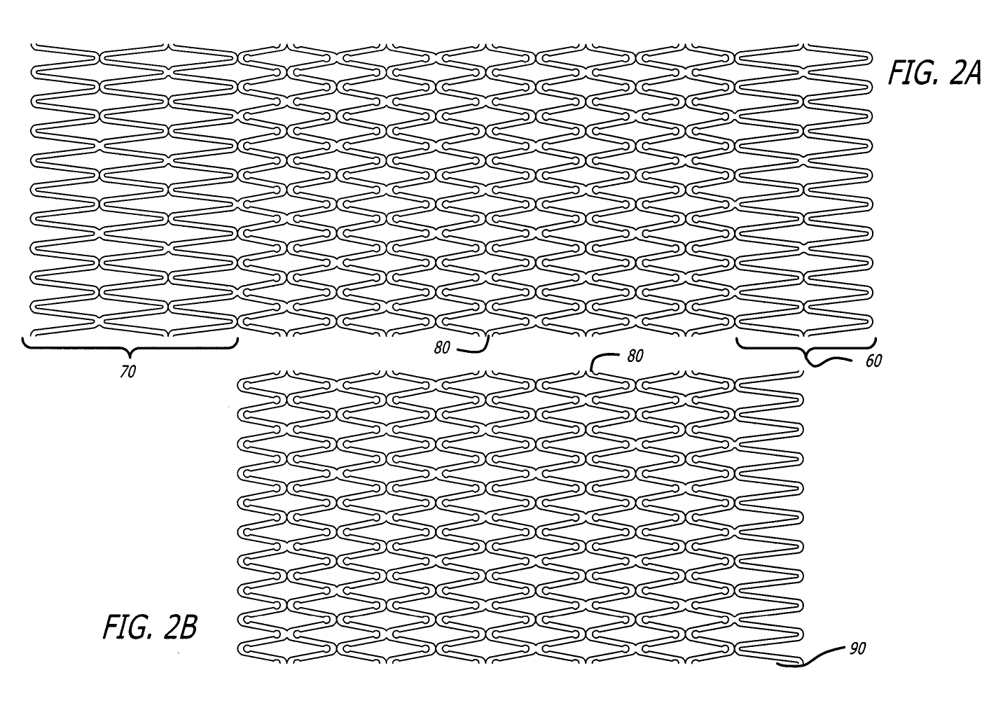

Referring to FIG. 3, in use, a balloon 14 of length B is fixed to catheter 12 using methods known to those skilled in the art. Balloon 14 is shown in its deflated and compressed state in FIG. 3. A stent 16 is mounted about balloon 14 by crimping and by two overlying retaining sleeves 18 and 20. Stent 16 is comprised of a body 30 and self-expanding nitinol end segments 32 and 34. Stent body 30 is crimped about balloon 14 while retaining sleeves 18 and 20 overly at least a portion of end segments 32 and 34. In an alternative embodiment, the retaining sleeves can overly all of the respective end segments and a portion of the stent body. In one embodiment of the present invention retaining sleeves 18 and 20 are silicone and have a length C and are axially fixed along catheter 12 by adhesive detents 22 and 24 formed of a urethane bead. Distal detent 22 can be tapered to ensure that the catheter assembly does not damage or traumatize a body lumen as it is advanced to the treatment (deployment) site. Thus, the catheter tip, the distal detent 22 and distal sleeve 20 can form a continuously tapered surface. Proximal sleeve detent 22 can also be tapered if desired. Further, sleeves 18 and 20 may have tapered edges. The sleeves overlap at least a portion of the self-expanding nitinol end segments 32 and 34 at each end or margin of stent 16, and can overlap all of the end segments and a portion of the stent body. A detailed prior art example of stent deployment useful with the teachings of the present invention, can be found in U.S. Pat. No. 4,950,227 to Savin et al. which is hereby incorporated by reference in its entirety.

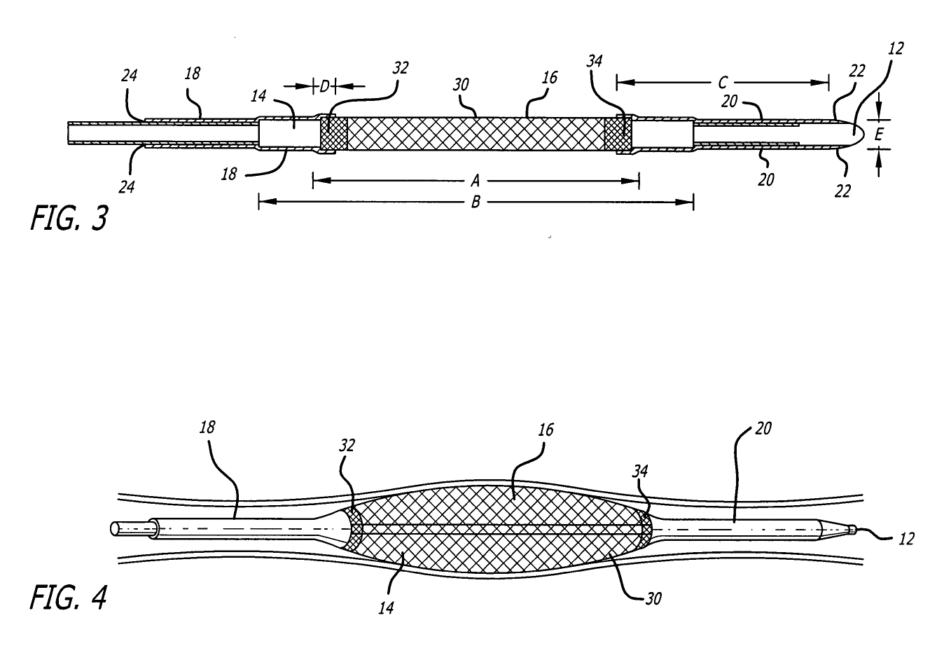

During deployment of the stent 16 of the present invention, the balloon 14 is expanded to deploy the body 30 of the stent 16. As shown in FIG. 4, expansion of the balloon 14 causes sleeves 18 and 20 to fold back. Once sleeves 18 and 20 fold back due to balloon 14 expansion, the self-expanding nitinol end segments 32 and 34 begin to be more exposed, and when sufficiently more exposed by the sleeves 18 and 20 will deploy and stent a portion of the vessel. The length of balloon 14 can be varied with respect to the length of stent 16 such that that portion of the lumen supported by end segments 32 and 34 is not significantly affected by balloon 14 expansion. After deployment of the body 30 and ends 32 and 34 of the stent 16 in this manner, the stent 16 is released from sleeves 18 and 20, which then contract about balloon 14 when it is deflated. This deflation allows removal of balloon 14 and sleeves 18 and 20 with the catheter 12, as the catheter 12 is axially removed from the vessel. If required, a lubricating solution can be provided between balloon 14 and sleeves 18 and 20 to aid in release of stent 16 from the sleeves.

Adhesive detents 22 and 24 may be replaced with fixed detents formed along catheter 12 during its manufacture by methods known to those having ordinary skill in the art. These detents act as stoppers to prevent axial dislodgement of sleeves 18 and 20. Further, sleeves 18 and 20 may be affixed to the catheter with an underlying adhesive or overlaying shrink tube, a crimped metal ring, or a suture or other methods known in the art.

While sleeves 18 and 20 of the present invention have been described as made of silicone, these sleeves can be formed of any other expandable substance, for example, polyurethane, latex or polyether amide. Generally, any elastomeric memory material able to expand by at least two times its at rest diameter and return to the at rest state can be used in accordance with the teachings of the present invention. The material must also be able to be expanded at least two times by internal pressure below that usable with balloon angioplasty (about 3-17 atmospheres), and also be formable into a thin walled tube. Typical examples of such materials include, for example and without limitation, elastomers, such as natural rubber and thermoplastic elastomers, such as urethane, polyimides, and styrenes. Further, hydrophilic polymers are also suitable.

As stated earlier, the end segments of the stents of the present invention are self-expanding. In one embodiment, the body of the stent also can be self-expanding with sleeve 18 and 20 release allowing stent deployment to occur.

In one embodiment of the present invention the end segments comprise self-expanding nitinol end segments. Nitinol refers to a shape-memory material prepared from titanium-nickel alloys. The general properties of these nitinol shape-memory materials are related in an article by W. J. Buehler, et al., Wire Journal, June, 1969, pp. 41-49, and more extensively discussed in an article by McDonald Schetky, Scientific American, November, 1979, pp. 74-82. Briefly, shape-memory alloys have the property of mechanical “memory.” These materials can be formed into a first predetermined shape above a transition temperature range (TTR), the TTR being dependent on the particular ratio of metals in the alloy. Below the TTR the alloy is highly ductile and may be plastically deformed into a second desired shape. Upon reheating above the TTR the alloy returns to its first pre-set form.

In another embodiment of the present invention, the self-expanding nitinol end segments 32 and 34 can be comprised of a nitinol alloy that is approximately 55% nickel and 45% titanium. Self-expanding nitinol end segments 32 and 34 can be formed at a temperature above the TTR of the alloy into a first shape or configuration (i.e. deployed shape) and can be reformed into a second configuration (i.e. compressed shape) when cooled below the TTR of the alloy. Self-expanding nitinol end segments 32 and 34 can then be returned to their first configuration by exposure to a higher temperature above the alloy TTR. The temperatures at which these transitions occur are affected by the nature and condition of the nitinol alloy. In one embodiment of the present invention the transition temperature is designed to be slightly lower than body temperature. It can be desirable to have the transition temperature set at just below body temperature to enable a rapid transition when the stent is implanted in a body lumen.

In one embodiment, the nitinol alloys of the present invention can include at least one additional element selected from the group of elements consisting of palladium, platinum, chromium, iron, cobalt, vanadium, manganese, boron, copper, aluminum, tungsten, tantalum, or zirconium. In particular, the at least one additional element can optionally be up to approximately 3 percent each of iron, cobalt, platinum, palladium, and chromium, and up to about 10 percent copper and vanadium. In one embodiment of the present invention, the nitinol alloy can be formed from a composition consisting essentially of about 30 to about 52 percent titanium and the balance nickel and up to 10 percent of one or more of the above-mentioned additional alloying elements. As used herein, all references to percent composition are atomic percent unless otherwise noted (as used herein, “atomic percent” means the number of atoms of an element per unit volume divided by the number of atoms per unit volume of the substance containing the element).

As will be apparent to one of skill in the art, the configurations and lengths of the stent body and self-expanding end segments can be adjusted to meet particular treatment objectives. The components of the system of the present invention also can take various forms in relation to one another. For example, in one embodiment of the present invention, the self-expanding end segments can be made of the same material as the body of the stent. In another particular embodiment, the self-expanding end segments and body of the stent can be constructed of different materials. Further, in one embodiment of the present invention, the self-expanding end segments can be continuous with the body of the stent. In another embodiment, the self-expanding end segments and body of the stent can be two or more separate and distinct parts that have been attached. To improve positioning during use, radiopaque markers can be placed so as to mark the area of the stent where the body and self-expanding end segments meet.

The stents of the present invention can be coated with an appropriate material to alter their clinical performance. For instance, various coatings can be capable of releasing a drug or bioactive agent to assist in the repair of a diseased vessel and to assist in the prevention of restenosis. Further, as mentioned, the stents of the present invention can be coated with a material, such as a radiopaque dye or marker to allow for better positioning. These markers can be placed on the ends of the stents or to mark the location of the beginning and/or ends of the self-expanding end segments of the stents. The coating of the present invention also can be continuous or discontinuous on the surface of the stents and can be disposed on the interior and/or the exterior surface(s) of the stents. Coatings can include one or more layers and can be coated either directly onto the stents or onto a primer material on the stents.

Any coating placed on the stents of the present invention should be biocompatible in order to minimize adverse interaction with the walls of the vessel or duct lumen or with the liquid flowing through the lumen. The coating can consist of polymeric or nonpolymeric coating materials. Non-limiting examples of suitable polymers can be found in published International Patent Application Publication Nos. WO-A-93/16479 and WO-A-93/15775 which are hereby incorporated by reference for all they contain with regard to coating materials. Further, non-limiting examples of non-polymer coatings suitable for use in accordance with the teachings of the present invention are disclosed in co-owned U.S. patent application Ser. No. 10/196,296 to Gertner et al., the entire contents of which is hereby incorporated herein by reference.

Many substances that can enhance clinical performance can be included in coatings of the stent of the present invention. Drugs and bioactive agents that can enhance the clinical performance of the stent of the present invention also can be included. Examples of such drugs and bioactive agents include, for example and without limitation, antineoplastic, antinflammatory, antiplatelet, anticoagulant, antifibrin, antithromobin, antimitotic, antibiotic, antiproliferative and antioxidant substances, as well as calcium channel blockers, colchicine fibroblast growth factor antagonists, histamine antagonists, 3-hydroxy-3-methylglutaryl coenzyme A (HMG-CoA) reductase inhibitors, monoclonal antibodies, phosphodiesterase inhibitors, prostaglandin inhibitors, platelet-derived growth factor antagonists, serotonin inhibitors, steroids, and thioprotease inhibitors. Additional substances can include, for example and without limitation, paclitaxel and its derivatives, rapamycin and its derivatives, cladribine, heparin, nitrous oxide, nitric oxide, actinomycin D, as well as, alpha-interferon, genetically engineered epithelial cells, and fish oil (omega 3-fatty acid).

Although the present invention has been discussed most thoroughly in relation to its use in the coronary vasculature, it may be used within any lumen within the body. A physician may position a stent 16 of the present invention at a treatment site and expand the balloon 14 by standard techniques. During this expansion, stent 16 is expanded to fill the lumen of the treatment site. Sleeves 18 and 20 release stent 16, at which point balloon 14 is deflated by standard techniques. Catheter 12 and sleeves 18 and 20 are then axially removed from the lumen while stent 16 remains in place. Based on this generalized description, the stents of the present invention can be used in any blood vessel, including, for example and without limitation, the coronary vasculature (which includes without limitation the right, left common, left anterior descending and circumflex arteries and their branches) and the peripheral vasculature (including without limitation branches of the carotid, aorta, femoral, renal, popliteal, and related arteries). While the stents of the present invention mainly have been described in terms of their use in a blood vessel, they can also be used in other lumens of the body, for example and without limitation, respiratory ducts, gastrointestinal ducts, bile ducts, the urinary system, the digestive tube, and the tubes of the reproductive system in both men and women.

It is to be understood that the present invention is not limited to the particular embodiments, materials, and examples described herein, as these can vary. It also is to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a stent” or “a self-expanding nitinol end segment” is a reference to one or more stents or self-expanding nitinol end segments and includes equivalents thereof known to those skilled in the art and so forth.

Unless defined otherwise, all technical terms used herein have the same meanings as commonly understood by one of ordinary skill in the art to which this invention belongs. Specific methods, devices, and materials are described, although any methods and materials similar or equivalent to those described herein can be used in the practice or testing of the present invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A stent comprising a body portion wherein said body portion has a first end and a second end and wherein said first end is attached to a first end segment and wherein said body portion comprises a first material and said first end segment comprises a second material which is different from said first material and wherein said second material is a self-expanding material.

2. The stent according to claim 1, wherein said second end of said body portion is attached to a second end segment that comprises a self-expanding material.

3. The stent according to claim 1, wherein said first material is selected from the group consisting of stainless steel, titanium, gold, cobalt alloys, magnesium, platinum, platinum alloys and tantalum alloys.

4. The stent according to claim 1, wherein said self-expanding material is a metal alloy selected from the group consisting of CuZnAl, CuAlNi, spring temper stainless steel and nickel-titanium alloys.

5. The stent according to claim 4 wherein said nickel-titanium alloy is nitinol.

6. The stent according to claim 2, wherein said self-expanding material is a metal alloy selected from the group consisting of CuZnAl, CuAlNi, spring temper stainless steel and nickel-titanium alloys.

7. The stent according to claim 1, further comprising a coating, said coating comprising at least one biocompatible polymer or metal and within said biocompatible polymer or metal, a bioactive agent selected from the group consisting of antineoplastic agents, antinflammatory agents, antiplatelet agents, anticoagulant agents, antifibrin agents, antithromobin agents, antimitotic agents, antibiotic agents, antiproliferative agents, antioxidant substances, calcium channel blockers, colchicine fibroblast growth factor antagonists, histamine antagonists, 3-hydroxy-3-methylglutaryl coenzyme A (HMG-CoA) reductase inhibitors, monoclonal antibodies, phosphodiesterase inhibitors, prostaglandin inhibitors, platelet-derived growth factor antagonists, serotonin inhibitors, steroids, and thioprotease inhibitors.

8. A system for deploying a stent comprising: a catheter, said catheter having a catheter shaft; said catheter shaft having a distal end and a proximal end; an inflatable balloon portion disposed within said distal end of said catheter shaft and in fluid communication with said catheter shaft such that said inflatable balloon portion can be inflated; said inflatable balloon portion having a proximal end and a distal end; a stent disposed over said inflatable balloon portion, said stent comprising a body portion wherein said body portion has a first end and a second end and wherein said first end is attached to a first end segment and wherein said body portion comprises a first material and said first end segment comprises a second material wherein said second material is a self-expanding material.

9. The system according to claim 8, wherein said second end of said body portion is attached to a second end segment that comprises a self-expanding material.

10. The system according to claim 8 wherein said first material is not a self-expanding material.

11. The system according to claim 8, wherein said stent further comprises a coating, said coating comprising at least one biocompatible polymer or metal and within said biocompatible polymer or metal, a bioactive agent selected from the group consisting of antineoplastic agents, antinflammatory agents, antiplatelet agents, anticoagulant agents, antifibrin agents, antithromobin agents, antimitotic agents, antibiotic agents, antiproliferative agents, antioxidant substances, calcium channel blockers, colchicine fibroblast growth factor antagonists, histamine antagonists, HMG-CoA reductase inhibitors, monoclonal antibodies, phosphodiesterase inhibitors, prostaglandin inhibitors, platelet-derived growth factor antagonists, serotonin inhibitors, steroids, and thioprotease inhibitors.

12. The system according to claim 8, wherein said first end segment of said stent extends beyond said proximal end of said balloon and said second end segment extends beyond said distal end of said balloon.

13. The system according to claim 8, wherein said first end segment and said second end segment are held in contact with said catheter by retaining sleeves.

14. The system according to claim 13, wherein said retaining sleeves are affixed to said catheter using an adhesive.

15. A method for providing a system for deploying a stent comprising:
providing a catheter, said catheter having a catheter shaft with a distal end and a proximal end;
providing an inflatable balloon portion disposed within said distal end of said catheter shaft and in fluid communication with said catheter shaft such that said inflatable balloon portion can be inflated, said inflatable balloon portion having a proximal end and a distal end;
disposing a stent over said inflatable balloon portion, said stent comprising a body portion wherein said body portion has a first end and a second end and wherein said first end is attached to a first end segment and wherein said body portion comprises a first material and said first end segment comprises a second material wherein said second material is a self-expanding material; and wherein said stent further comprises a bioactive material-containing coating wherein said coating is a metal and said bioactive material is paclitaxel.

16. The method according to claim 15, wherein said second end of said body portion is attached to a second end segment that comprises a self-expanding material.

17. The method according to claim 15, wherein said first end segment of said stent extends beyond said proximal end of said balloon and said second end segment extends beyond said distal end of said balloon.

18. The method according to claim 15, wherein said first end segment and said second end segment are held in contact with said catheter by silicone retaining sleeves.

19. The method according to claim 18, further comprising a lubricant associated with said retaining sleeves.

20. The method according to claim 18, wherein said retaining sleeves are affixed to said catheter using an urethane-based adhesive.

21. The method according to claim 15, wherein said first material is selected from the group consisting of stainless steel, titanium, gold, cobalt alloys, magnesium, platinum, platinum alloys and tantalum alloys.

22. The method according to claim 15, wherein said self-expanding material is a metal alloy selected from the group consisting of CuZnAl, CuAlNi, spring temper stainless steel and nickel-titanium alloys.
